(19) United States
(12) Patent Application Publication
(10) Pub. No.: US 2025/0059125 A1
(43) Pub. Date: Feb. 20, 2025

(54) METHOD OF PRODUCING ANTHRAQUINONE-BASED SUBSTANCE

(71) Applicants: MITSUBISHI HEAVY INDUSTRIES, LTD., Tokyo (JP); NAGOYA DENKI EDUCATIONAL FOUNDATION, Aichi (JP)

(72) Inventors: Koichiro Hirayama, Tokyo (JP); Toshiyasu Kiyabu, Tokyo (JP); Yasushi Morita, Aichi (JP); Tsuyoshi Murata, Aichi (JP); Aya Ito, Aichi (JP); Shigemitsu Okada, Aichi (JP)

(73) Assignees: MITSUBISHI HEAVY INDUSTRIES, LTD., Tokyo (JP); NAGOYA DENKI EDUCATIONAL FOUNDATION, Aichi (JP)

(21) Appl. No.: 18/721,371

(22) PCT Filed: Dec. 19, 2022
(86) PCT No.: PCT/JP2022/046572

§ 371 Date: Jun. 18, 2024

(30) Foreign Application Priority Data
Dec. 22, 2021 (JP) 2021-208081

(51) Int. Cl. C07C 51/09

(52) U.S. Cl. CPC C07C 51/09

(57) ABSTRACT

A method of producing an anthraquinone-based substance represented by the following chemical formula:

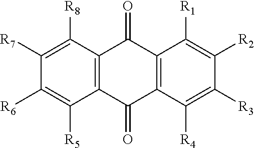

where at least one of R₁ to R₈ is a hydroxy group, and at least one of R₁ to R₈ is an alkoxy group, includes the steps of: preparing a starting material represented by the following chemical formula:

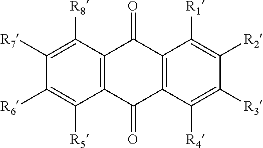

where at least two of the R₁′ to R₈′ are hydroxy groups; and reacting the starting material with an organic alkylating agent. The amount of the organic alkylating agent to be reacted with the starting material is more than or equal to 0.05 mol and less than n mol per 1 mol of the starting material, where n is the number of hydroxy groups contained in the starting material.

### TECHNICAL FIELD

The present disclosure relates to a method of producing an anthraquinone-based

substance.

The present application claims priority based on Japanese Patent Application No. 2021-208081 filed on Dec. 22, 2021, the entire content of which is incorporated herein by reference.

### BACKGROUND ART

Redox flow batteries are suitable for storing a large amount of electricity because the electricity storage amount can be freely designed according to the tank capacity, and they are expected to be applied to leveling the demand for electricity, including natural energy. Redox flow batteries are characterized in that they are composed of a cell that charges and discharges and an electrolyte tank for power storage, and a pump is used to circulate electrolyte for charging and discharging.

Currently, redox flow batteries that use vanadium as an active material in electrolyte

are the mainstream, but due to the recent surge in vanadium prices and other factors, redox flow batteries that use organic materials or metal complexes as the active material are being developed. For example, Patent Document 1 describes a redox flow battery that uses anthraquinone or naphthoquinone as a negative electrode active material, and a number of anthraquinones with sulfo groups are illustrated. Patent Document 2 describes, although not an active material itself, a redox flow battery that uses as the active material a composition containing a coordination compound with a redox non-innocent ligand coordinated to a metal center, and as the redox non-innocent ligand, a number of anthraquinones with various functional groups bonded to the 1-to 8-positions of anthraquinones are illustrated. Non-Patent Documents 1 and 2 also describe compounds with various functional groups or elements bonded to the 1-to 8-positions of anthraquinones.

### Citation List

### Patent Literature

Patent Document 1: JP6574382B

Patent Document 2: JP2019-514170A (translation of a PCT application)

### Non-Patent Literature

Non-Patent Document 1: K. Lin, Q. Chen, M. R. Gerhardt, L. Tong, S. B. Kim, L. Eisenach, A. W. Valle, D. Hardee, R, G. Gordon, M, J. Aziz, M. P. Marshak, Science, 349 (2015) 1529-1532Non-Patent Document 2: D. G. Kwabi, K. Lin, Y. Ji. F. Kerr, M. Goulet, D. D. Porcellinis, D. P. Tabor, D. A. Pollack, A. Aspuru-Guzik, R. G. Gordon, M. J. Aziz, Joule 2, 19 (2018) 1894-1906

SUMMARY

### Problems to be Solved

Important parameters for active materials in redox flow batteries include redox potential, solubility, durability, and electrolyte viscosity. In order to bring these parameters close to ideal values, it is important to select an appropriate substituent attached to each of the 1-to 8-positions of anthraquinone. In Patent Document 1 and Non-Patent Documents 1 and 2, the substituent attached to each of the 1-to 8-positions of anthraquinone can be independently selected, but no method has been established for practical synthesis of anthraquinone-based substances with specific combinations of substituents introduced, with the exception of a few compounds.

In view of the above, an object of at least one embodiment of the present disclosure is to provide a method of producing an anthraquinone-based substance whereby it is possible to efficiently obtain an anthraquinone-based substance having different substituents.

### Solution to the Problems

In order to achieve the above object, a method of producing an anthraquinone-based substance represented by the following chemical formula:

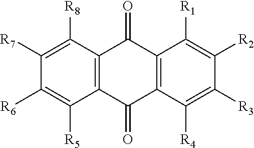

where at least one of the R₁ to R₈ is a hydroxy group, and at least one of the R₁ to R₈ is an alkoxy group, includes the steps of: preparing a starting material represented by the following chemical formula:

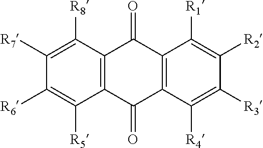

where at least two of the R₁′ to R₈′ are hydroxy groups; and reacting the starting material with an organic alkylating agent. The amount of the organic alkylating agent to be reacted with the starting material is more than or equal to 0.05 mol and less than n mol per 1 mol of the starting material, where n is the number of hydroxy groups contained in the starting material.

### Advantageous Effects

With the method of producing an anthraquinone-based substance of the present disclosure, by the reaction of more than or equal to 0.05 mol and less than n mol of the organic alkylating agent per 1 mol of the starting material, where n is the number of hydroxy groups contained in the starting material, hydroxy groups in the molecule of the starting material partially react with the organic alkylating agent, so that an anthraquinone-based substance with different substituents can be efficiently obtained.

### BRIEF DESCRIPTION OF DRAWINGS

FIG. 1 is a flowchart for describing a method of separating a monosubstituted product from a reaction solution obtained by chemical reaction formula (6).

FIG. 2 is a flowchart for describing another method of separating a monosubstituted product from a reaction solution obtained by chemical reaction formula (6).

FIG. 3 is a flowchart for describing a method of separating a monosubstituted product from a product obtained by chemical reaction formula (7).

### DETAILED DESCRIPTION

Hereinafter, the method of producing an anthraquinone-based substance according to embodiments of the present disclosure will be described with reference to the drawings. The embodiment to be described below indicates one aspect of the present disclosure, does not intend to limit the disclosure, and can optionally be modified within a range of a technical idea of the present disclosure.

### <Method of Producing Anthraquinone-Based Substance>

The anthraquinone-based substance obtained by the production method described below is an anthraquinone-based substance represented by the following chemical formula (1), where at least one of R₁ to R₈ bonded to the 1- to 8-positions of the anthraquinone skeleton is a hydroxy group, and at least one of R₁ to R₈ is an alkoxy group. That is, an anthraquinone-based substance having different substituents, hydroxy and alkoxy groups, is produced.

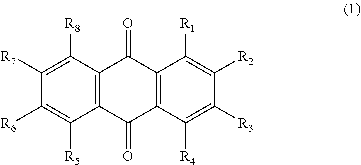

In this production method, a compound represented by the following chemical formula (2) is used as a starting material. In chemical formula (2), at least two of R₁′ to R₈′ bonded to the 1- to 8-positions of the anthraquinone skeleton are hydroxy groups.

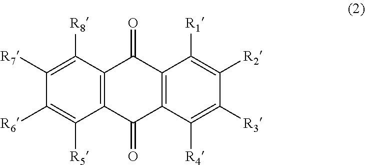

In this production method, the starting material is reacted with an organic alkylating agent (RX) in the presence of a base, as shown in the following chemical reaction formula (3). In chemical reaction formula (3), R is an alkyl group, and X is any desorbing group such as halogen, tosylate, mesylate, sulfonate, or phosphonate. R has 1 to 6 carbon atoms, and when R has 4 to 6 carbon atoms, it has a linear or branched structure. The bond between carbon atoms constituting R is not limited to a single bond, but may include a double or triple bond. R may contain an ether bond. Further, at least one of the carbon atoms constituting R may be bonded to a halogen or any functional group, such as a sulfo group, amino group, nitro group, carboxy group, phosphoryl group, thiol group, or alkyl ester, instead of hydrogen. As the base, NaH, NaOH, KOH, or K₂CO₃ can be used.

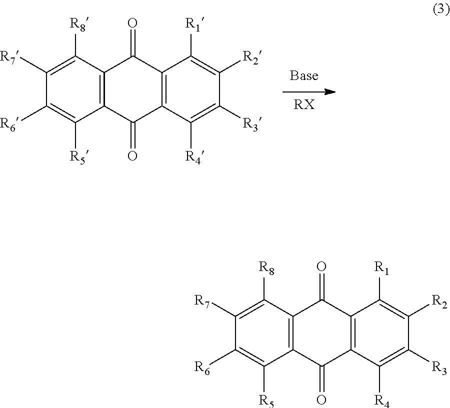

In chemical reaction formula (3), the amount of the organic alkylating agent to be reacted with the starting material is preferably more than or equal to 0.5 mol and less than n mol per 1 mol of the starting material, where n is the number of hydroxy groups contained in the starting material. With this condition, hydroxy groups in the molecule of the starting material partially react with the organic alkylating agent, so that an anthraquinone-based substance with different substituents, i.e., hydroxy and alkoxy groups, can be efficiently obtained. However, even when the amount of the organic alkylating agent is less than 0.5 mol per mol of the starting material, this effect can still be achieved, although more work is required to collect the product. However, considering the increased product collection efforts, the amount of the organic alkylating agent may be more than or equal to 0.05 mol and n mol per 1mol of the starting material.

For example, when 2,6-dihydroxyanthraquinone (2,6-DHAQ) is used as the starting material in this production method, as shown in the following chemical reaction formula (4), for example, the reaction between 0.5 mol to 1.5 mol of the organic alkylating agent RX and 1mol of 2,6-DHAQ produces an anthraquinone-based substance with a hydroxy group (OH) attached to the 2-position and an alkoxy group (OR) attached to the 6-position and an anthraquinone-based substance with an alkoxy group attached to the 2-position and a hydroxy group attached to the 6-position. In addition to those, unreacted starting material and an anthraquinone-based substance with alkoxy groups attached to the 2-and 6-positions may also be present, yielding a mixture of the four types of anthraquinone-based substances.

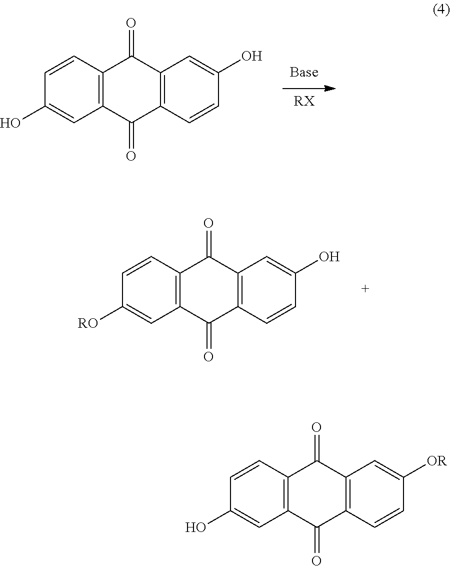

Furthermore, for example, when 2,3,6,7-tetrahydroxyanthraquinone (2,3,6,7-THAQ) is used as the starting material in this production method, since the starting material contains four hydroxy groups, the amount of the organic alkylating agent to be reacted with the starting material is more than or equal to 1 mol and less than 4 mol per 1 mol of the starting material. This production method yields a mixture of anthraquinone-based substances (Compounds 1 to 5) with combinations of substituents at the 2-, 3-, 6-, and 7-positions as shown in Table 1 below, starting material, and anthraquinone-based substance with alkoxy groups attached to all of the 2-, 3-, 6-, and 7-positions.

TABLE 1

| Compound | 2 - position | 3 - position | 6 - position | 7 - position |
|---|---|---|---|---|
| 1 | OR | OH | OH | OH |
| 2 | OR | OR | OH | OH |
| 3 | OR | OH | OR | OH |
| 4 | OR | OH | OH | OR |
| 5 | OR | OR | OR | OH |

### <Variation of Production Method of Present Disclosure>

In this production method, an alkyl carboxylic acid ester halide may be used as the organic alkylating agent. In this case, the alkyl carboxylic acid ester halide can be represented by a general formula X—Rₐ—COOR_{b}, and R of the organic alkylating agent represented by RX corresponds to Rₐ—COOR_{b}. In this general formula, Rₐ and R_{b} are any alkyl groups. For example, the reaction of 2,6-DHAQ, the starting material, with the alkyl carboxylic acid ester halide produces an intermediate anthraquinone-based substance in which hydrogen of a hydroxy group is replaced by Rₐ—COOR_{b}, as in the first-step reaction in the following chemical reaction formula (5). In the next second-step reaction, ester hydrolysis occurs by reaction of this intermediate in the presence of a base, and treatment with an acid results in the alkoxy group terminating in a carboxy group. With this production method, it is possible to efficiently obtain an anthraquinone-based substance having an alkoxy group containing a carboxy group.

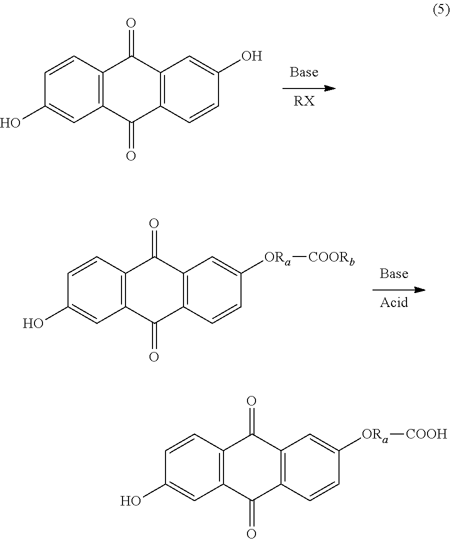

For example, in this production method, ethyl 4-bromobutanoate can be used as the alkyl carboxylic acid ester halide (corresponding to Rₐ=C₃H₆ and R_{b}=C₂H₅(Et)). In this case, the reaction of 2,6-DHAQ, the starting material, with ethyl 4-bromobutanoate produces an intermediate anthraquinone-based substance in which hydrogen of one hydroxy group is replaced by a 3-(ethoxycarbonyl) propyl group, as represented by the following chemical reaction formula (6). Then, ester hydrolysis occurs by reaction of this intermediate in the presence of a base, and treatment with acetic acid (AcOH) yields an anthraquinone-based substance (2-(3′-carboxypropyloxy)-6-hydroxy-9,10-anthraquinone (2,6-(MHMBEAQ)) having one hydroxy group and one alkoxy group containing a carboxy group.

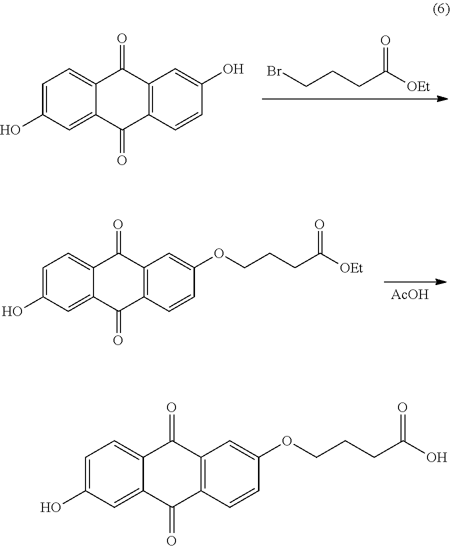 [SMILES: "AcOH" ; O=C1c2ccc(O)cc2C(=O)c2ccc(O)cc21 ; CCOC(=O)CCCBr ; CCOC(=O)CCCOc1ccc2c(c1)C(=O)c1ccc(O)cc1C2=O ; O=C(O)CCCOc1ccc2c(c1)C(=O)c1ccc(O)cc1C2=O]

If the carbon chain of the organic alkylating agent is short, E2 reaction may occur, causing the organic alkylating agent to partially decompose and no longer contribute to the reaction. In contrast, when ethyl 4-bromobutanoate is used as the organic alkylating agent, since the carbon chain length of the butanoic acid backbone of ethyl 4-bromobutanoate is of an appropriate length, ethyl 4-bromobutanoate is difficult to decompose. Therefore, most of the ethyl 4-bromobutanoate added can contribute to the reaction, and the yield of the target product can be increased.

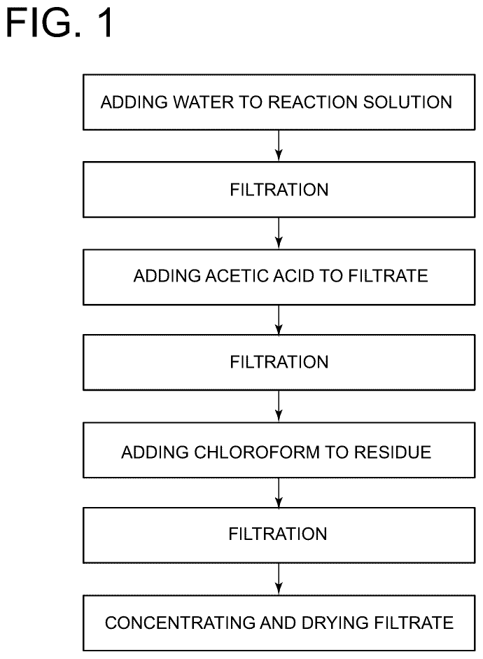

In chemical reaction formula (6), the formation of an anthraquinone-based substance with one hydroxy group and one alkoxy group attached (hereinafter referred to as “monosubstituted product”) is shown, but in reality, a mixture with unreacted starting material and an anthraquinone-based substance with two alkoxy groups attached (hereinafter referred to as “disubstituted product”) is obtained. In the following, two methods for separating the monosubstituted product from this mixture obtained by the production method will be described. A flowchart of the first method is shown in FIG. 1. Water is added to the reaction

solution of chemical reaction formula (6). When the reaction solution is filtered, the filtrate contains mainly the monosubstituted product and unreacted starting material. On the other hand, the residue contains mainly the disubstituted product and monosubstituted product. Since the filtrate from this filtration is alkaline, acetic acid is added to neutralize the filtrate, followed by a second filtration. Chloroform is added to the residue from the second filtration, followed by a third filtration. The monosubstituted product can be separated by concentrating and drying the filtrate from the third filtration.

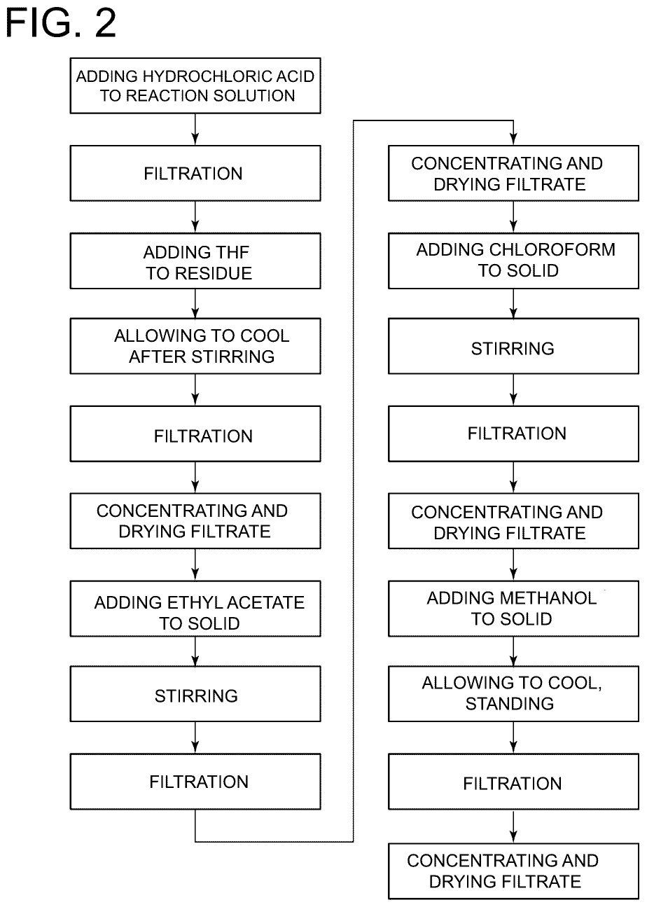

A flowchart of the second method is shown in FIG. 2. Since the reaction solution

of chemical reaction formula (6) is alkaline, hydrochloric acid is added to neutralize the solution. The neutralized reaction solution is filtered. Tetrahydrofuran (THF) is added to the residue and stirred for 4 hours in the temperature range of 40 to 60° C., then allowed to cool. The resulting mixture is filtered, and the filtrate is concentrated and dried. Ethyl acetate is added to the solid obtained by concentration and drying and stirred for 1 hour in the temperature range of 40 to 70° C. The mixture is then filtered in the temperature range from room temperature to 50° C. The filtrate is concentrated and dried. Chloroform is added to the solid obtained by concentration and drying and stirred for 1 hour in the temperature range of 40 to 60° C. The resulting mixture is filtered, and the filtrate is concentrated and dried. Methanol is added to the solid obtained by concentration and drying and heated to 45 to 55° C. The mixture is then allowed to cool and stand still. The monosubstituted product can be separated by filtering the cooled mixture and concentrating and drying the filtrate. The stirring time in the above explanation is not exact but approximate, and can be changed as long as it is not significantly shorter or longer than the time described above.

According to the inventors' experiments in this disclosure, it was confirmed that the

reaction represented by chemical reaction formula (6) yielded a reaction solution containing about 40 mass % monosubstituted product, about 30 mass % disubstituted product, and about 30 mass % unreacted starting material. When the two methods were performed on this reaction solution to separate the monosubstituted product, the yield of the monosubstituted product was 12 to 18% in the first method, whereas the yield of the monosubstituted product was about 40% in the second method. In the first method, since the residue from the first filtration contains the monosubstituted product, the amount of monosubstituted product that cannot be collected increases by the amount of monosubstituted product contained in the residue. Both methods can be used to separate the monosubstituted product, but the second method is the preferred method because of its higher yield.

The hydrolysis reaction of chemical reaction formula (6) is carried out in an organic solvent such as isopropyl alcohol or 1,2-dimethoxyethane in the presence of a base, but the hydrolysis can be carried out without such an organic solvent by using potassium hydroxide or sodium hydroxide aqueous solution (alkaline aqueous solution). In this case, subsequent treatment with an acid can be omitted. Also, even if the hydrolysis is performed using an organic solvent, the organic solvent can be distilled off after the hydrolysis reaction by vacuum concentration without acid treatment. In this case, an alkaline aqueous solution is obtained. When hydrolyzed in this manner, the hydroxy group of the final product in chemical reaction formula (6) becomes —OK or —ONa, and the carboxy group becomes —COOK or —COONa. When a compound having hydroxy and carboxy groups, such as 2,6-MHMBEAQ, is dissolved in alkaline electrolyte and used as an active material, alkali is consumed to neutralize the hydroxy and carboxy groups. To prepare alkaline electrolyte for use in redox flow batteries, additional alkali is required to neutralize the hydroxy and carboxy groups. However, a solution in which the final product obtained by the hydrolysis using potassium hydroxide or sodium hydroxide dissolves can be used as-is in redox flow batteries as alkaline electrolyte.

The production method of the present disclosure has been specifically described using ethyl 4-bromobutanoate as an example of the alkyl carboxylic acid ester halide, but instead of the ethyl group constituting the ester group, alkyl 4-bromobutyric acid with a methyl group, propyl group, butyl group, or any other alkyl group attached to the carboxy group may be used. However, it is preferable to use alkyl 4-bromobutanate with an alkyl group having a carbon chain with four or more carbon atoms bonded in a branched manner in the ester group. The use of such alkyl carboxylic acid ester halide facilitates separation of anthraquinone-based substances in the washing and extraction processes due to a large difference in solubility between an anthraquinone-based substance with different substituents and an anthraquinone-based substance with identical substituents, which are by-products.

For example, as represented by the following chemical reaction formula (7), the starting material, 2,6-DHAQ, is reacted with (2′-ethyl) hexyl 4-bromobutanate as the alkyl carboxylic acid ester halide. This produces a monosubstituted product in which one of the two hydroxy groups is replaced by an alkoxy group and a disubstituted product in which both of the two hydroxy groups are replaced by alkoxy groups. The reaction solution obtained by chemical reaction formula (7) also contains unreacted starting material in addition to the monosubstituted and disubstituted products.

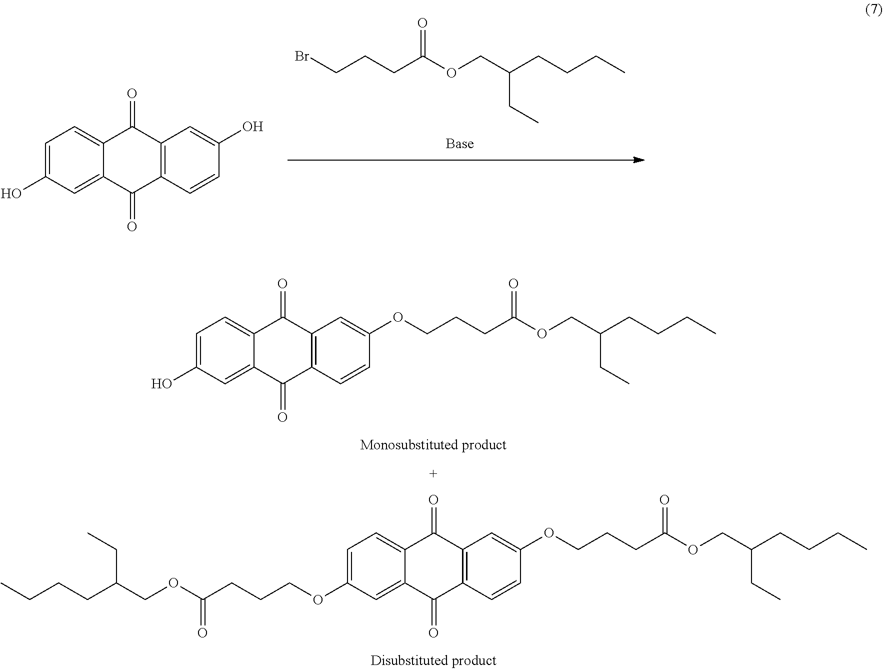

Next, a method for separating the monosubstituted product from the reaction solution obtained by chemical reaction formula (7) will be described based on the flowchart of

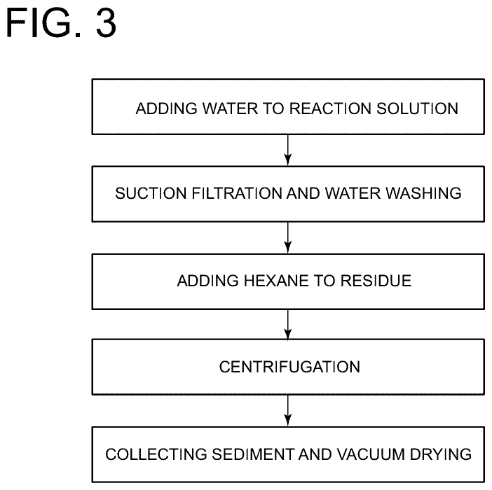

FIG. 3. Water is added to the reaction solution, followed by suction filtration and washing with water. Hexane is added to the resulting residue, and the mixture is centrifuged. Since the monosubstituted product and the disubstituted product have significantly different solubilities in hexane, the liquid phase obtained by centrifugal separation contains mainly the disubstituted product dissolved, while the sediment obtained by centrifugal separation contains mainly the monosubstituted product. The monosubstituted product can be separated by collecting and vacuum drying the sediment. Hydrolysis of this monosubstituted product produces 2,6-MHMBEAQ, in which the 2-ethylhexyl group constituting the ester group is replaced by a hydrogen atom, at high yield.

Since 2,6-DHAQ is industrially mass-produced, it is readily available as the starting material, thus reducing the cost of producing the target anthraquinone-based substance. On the other hand, 2,6-DHAQ can be easily synthesized from 2,6-diaminoanthraquinone (2,6-DAAQ) by the known Sandmeyer reaction. Since 2,6-DAAQ is less expensive than 2,6-DHAQ (approximately one-tenth or less), 2,6-DHAQ synthesized from 2,6-DAAQ by the

Sandmeyer reaction may be used as the starting material to further reduce the production cost of the target anthraquinone-based substance.

### (EXAMPLES)

### <Example 1>

2,6-MHMBEAQ was synthesized from 2,6-DHAQ by the procedure represented by the following chemical reaction formula (8). The synthesis is outlined as follows: from 2,6-DHAQ as the starting material, an intermediate with an alkoxy group in which hydrogen of one hydroxy group is replaced by ethyl butanoate is synthesized, and from this intermediate, the target substance, 2,6-MHMBEAQ, is synthesized.

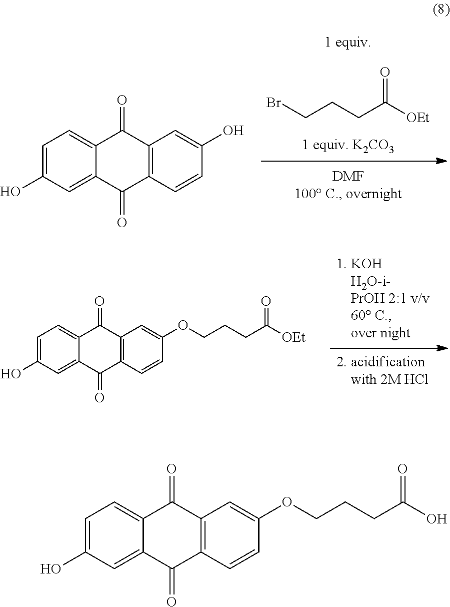 [SMILES: O=C1c2ccc(O)cc2C(=O)c2ccc(O)cc21 ; CCOC(=O)CCCBr ; CCOC(=O)CCCOc1ccc2c(c1)C(=O)c1ccc(O)cc1C2=O ; O=C(O)CCCOc1ccc2c(c1)C(=O)c1ccc(O)cc1C2=O]

In a 1 L eggplant flask, 40.0 g (167 mmol) of 2,6-DHAQ (Tokyo Chemical Industry Co., Ltd.) and 500 mL of N,N-dimethylformamide (DMF) were added, and 23.1 g (167 mmol) of potassium carbonate was added with stirring, followed by addition of 23.9 mL (167 mmol) of ethyl 4-bromobutanoate. The temperature rise was then started, and the mixture was stirred at 100° C. for 17 hours. After standing to cool, 600 mL of distilled water was added, the precipitate was suction filtered, and the residue was washed with distilled water. To the filtrate (pH>9), 6 M hydrochloric acid was added with stirring. After hydrochloric acid was added until the pH of the filtrate was less than 3 and carbon dioxide was no longer produced by addition of hydrochloric acid, the mixture was stirred at room temperature for 1 hour. The precipitate was transferred to a 200 mL centrifuge tube and centrifuged to separate the sediment.

The sediment was suction filtered and washed with distilled water, followed by vacuum drying at 80° C. for 6 hours to obtain 11.4 g of a mixture of raw material and intermediate. The resulting solid was ground to a powder and suspended in 200 mL of chloroform. Insoluble material was removed by suction filtration, and washing was performed with 200 mL of chloroform until soluble material was completely dissolved. Through this operation, 11.1 g of unreacted raw material was recovered. The filtrate was again suction filtered to completely remove insoluble material, and the filtrate was vacuum concentrated. The residue was suspended in distilled water, suction filtered, washed, and vacuum dried at 80° C. for 4 hours to obtain 6.96 g of intermediate as a reddish brown solid (12% yield).

Next, 6.96 g (19.6 mmol) of the intermediate was put in a 1L eggplant flask with 190 mL of isopropyl alcohol and 380 mL of distilled water. To this was added 4.48 g (79.9mmol) of potassium hydroxide, the temperature rise was started, and the mixture was stirred at 60° C. for 20 hours. After standing to cool, 550 mL of distilled water was added, the mixture was transferred to a 2L erlenmeyer flask, and 2 M hydrochloric acid was added with stirring until the pH was less than 3. After stirring for 2 hours, the sediment was separated by centrifugation. The supernatant and sediment were separately suction filtered, and the residue was washed with distilled water. The residue was vacuum dried at 80° C. for 4 hours to obtain 6.25 g of the target substance (98% yield from the intermediate). In Example 1, 1,2-dimethoxyethane can be used instead of isopropyl alcohol.

### <Example 2>

2,6-DHAQ was synthesized according to the following procedure by reaction represented by chemical reaction formula (9). In a 3L reaction vessel, 95.2 g (400 mmol) of 2,6-DAAQ (Tokyo Chemical Industry Co., Ltd.) and 1.6 L of 20% diluted sulfuric acid were placed. The mixture was stirred on an ice bath while 250 mL of an aqueous solution containing 71.8 g (1.04 mol) of sodium nitrite was added to the mixture for 1 hour. The mixture was stirred between -10° C. and-17° C. for 14 hours to obtain suspension of bisdiazonium salt. In another 5 L reaction vessel, 1.6 L of warm water was added, and the suspension of bisdiazonium salt was added to this warm water over about 2 hours while maintaining the temperature at 85 to 90° C.

After the suspension was completely added, stirring was continued for 2 hours while maintaining the same temperature. After standing to cool to room temperature, the precipitate was suction filtered, and the residue was washed with distilled water and heat dried for 15 hours to obtain 92.0 g of 2,6-DHAQ (96% yield). From 2,6-DHAQ thus obtained, 2,6-MHMBEAQ can be synthesized by the method of Example 1.

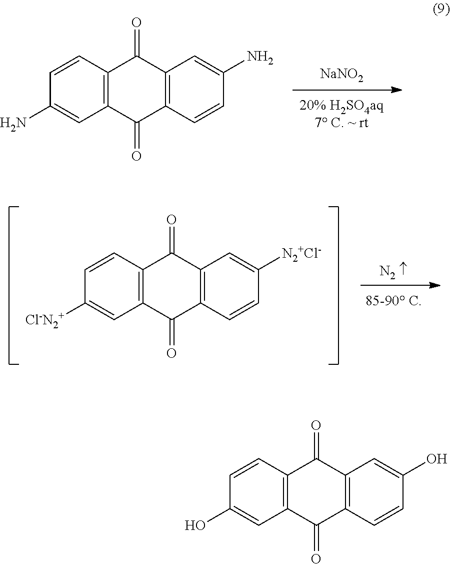

2,6-MHMBEAQ can also be synthesized by the method of the following two-step chemical reaction (10), instead of the method in Example 1.

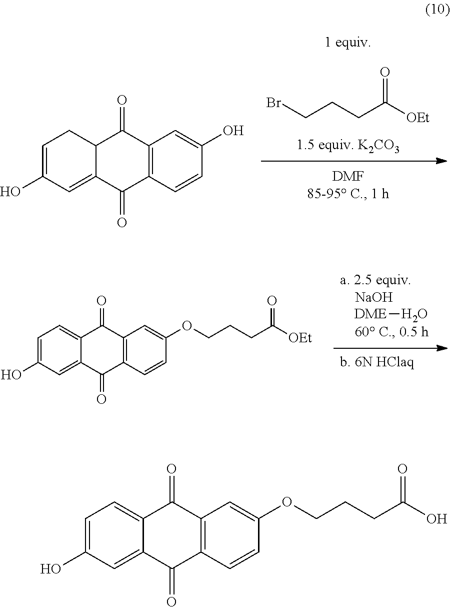

In a 3 L three-necked flask, 192 g (800 mmol) of 2,6-DHAQ and 2.2 L of DMF were placed. The temperature was raised to 85 to 95° C., and 84.0 g (608 mmol) of potassium carbonate was added with stirring. Then, 67.4 g (345 mmol) of ethyl 4-bromobutanoate was added, and stirring continued at this temperature for 1 hour. After standing to cool, 3 L of cold water (0 to 10° C.), 220 mL of 6M hydrochloric acid, and 2 L of water (15 to 30° C.) were added, and the precipitate was suction filtered, washed with water, and heat dried to obtain 217.7 g of a solid. To 150 g of this solid, 2.1 L of THF was added, the mixture was filtered to remove insoluble material, and the filtrate was concentrated to obtain 103 g of a solid. To this solid, 1.5 L of ethyl acetate was added, the mixture was filtered to remove insoluble material, and the filtrate was vacuum concentrated to obtain 72 g of a solid. To this solid, 1.1 L of chloroform was added, the mixture was filtered to remove insoluble material, and the filtrate was vacuum concentrated to obtain 52 g of a solid. To this solid, 800 mL of methanol was added, the mixture was filtered to remove insoluble material, and the filtrate was vacuum concentrated to obtain 36.6 g of a solid (21% yield in the first-step reaction).

In a 1 L eggplant flask, 25.0 g (71 mmol) of the solid obtained from the first-step reaction was placed, and 275 mL of ethylene glycol dimethyl ether was added to make a solution. To this was added 178 mL (178 mmol) of 1M sodium hydroxide aqueous solution, the temperature rise was started, and the mixture was stirred at 60° C. for 30 minutes. After allowing the reaction solution to cool, 33 mL of 6 M hydrochloric acid was added to acidify the solution, which was then vacuum concentrated to distill off the organic solvent. The precipitate was suction filtered, and the residue was washed with distilled water. The washed residue was air dried at 90° C. for 16 hours to obtain 22.2 g of 2,6-MHMBEAQ (96% yield in the second-step reaction).

### <Example 3>

By the reactions represented by the following chemical reaction formulas (11) to (15), a compound (target substance) with hydroxy groups attached to two of the 2-, 3-, 6-, and 7-positions of the anthraquinone skeleton and alkoxy groups (-OC3H6COOH) containing a carboxy group attached to the remaining two was synthesized according to the following procedure. First, 2,3,6,7-THAQ was synthesized according to the following procedure by the reactions represented by chemical reaction formulas (11) to (13).

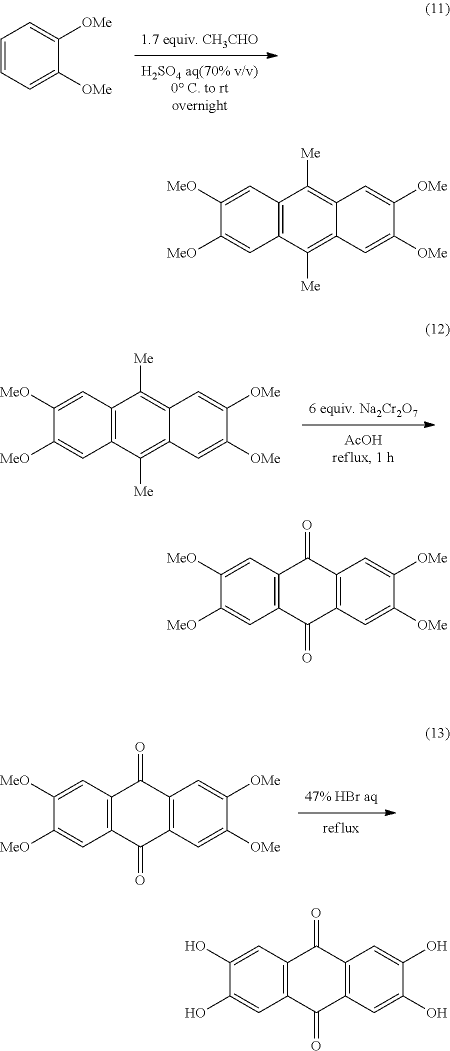 [SMILES: COc1ccccc1OC ; COc1cc2c(C)c3cc(OC)c(OC)cc3c(C)c2cc1OC ; COc1cc2c(C)c3cc(OC)c(OC)cc3c(C)c2cc1OC ; COc1cc2c(cc1OC)C(=O)c1cc(OC)c(OC)cc1C2=O ; COc1cc2c(cc1OC)C(=O)c1cc(OC)c(OC)cc1C2=O ; O=C1c2cc(O)c(O)cc2C(=O)c2cc(O)c(O)cc21]

In a 500 mL beaker, 42 g of ice and 100 mL of concentrated sulfuric acid were placed. A mixture solution of 25.1 g (182 mmol) of 1,2-dimethoxybenzene (available from Tokyo Chemical Industry Co., Ltd.) and 7.3 mL (308 mmol) of acetaldehyde was added dropwise to the reaction solution with stirring for 2.5 hours, taking care that the temperature of the reaction solution did not exceed 5° C. The solution was then stirred at room temperature for 22 hours. The resulting reaction solution was poured into a 1000 mL erlenmeyer flask containing 350 mL of ethanol and washed with 60 mL of methanol. The precipitate was suction filtered, and the residue was washed with 160 mL of ethanol and 320 mL of distilled water, followed by vacuum drying at 60° C. for 5 hours to obtain 22.3 g of a white solid (75% yield in chemical reaction formula (11)).

15.1 g (46.1 mmol) of the white solid was placed in a 1L eggplant flask and suspended in 750 mL of acetic acid, and 85.4 g (287 mmol) of sodium dichromate dihydrate was added. The reaction solution was heated and refluxed on an oil bath for 5 hours. After the reaction, the rection solution was allowed to cool and stand still, and the resulting precipitate was suction filtered. The residue was washed with distilled water, followed by vacuum drying at 70° C. for 4 hours to obtain 12.4 g of a yellow solid (82% yield in chemical reaction formula (12)).

18.8 g (57.1 mmol) of the yellow solid was placed in a 1L eggplant flask and suspended in 250 mL of 47% hydrobromic acid, and the suspension was heated and refluxed at 150° C. for 6 days on an oil bath. During the 6 days, 90 mL of 47% hydrobromic acid was added. After the reaction solution was allowed to cool, it was transferred to a centrifuge tube and centrifuged to remove the supernatant. The residue was dispersed by adding 400 mL of distilled water and centrifuged again to remove the supernatant. The insoluble material was suction filtered and then washed with distilled water, and the residue was vacuum dried at 70to 80° C. for 13 hours to obtain 15.1 g of 2,3,6,7-THAQ (98% yield in chemical reaction formula (13)).

From 2,3,6,7-THAQ thus obtained, the product was synthesized according to the following procedure by reaction represented by chemical reaction formula (14). The synthesis is outlined as follows: from 2,3,6,7-THAQ, an intermediate mixture with an alkoxy group in which hydrogen of two hydroxy groups is replaced by ethyl butanoate is synthesized, and from this intermediate mixture, the target substance is obtained.

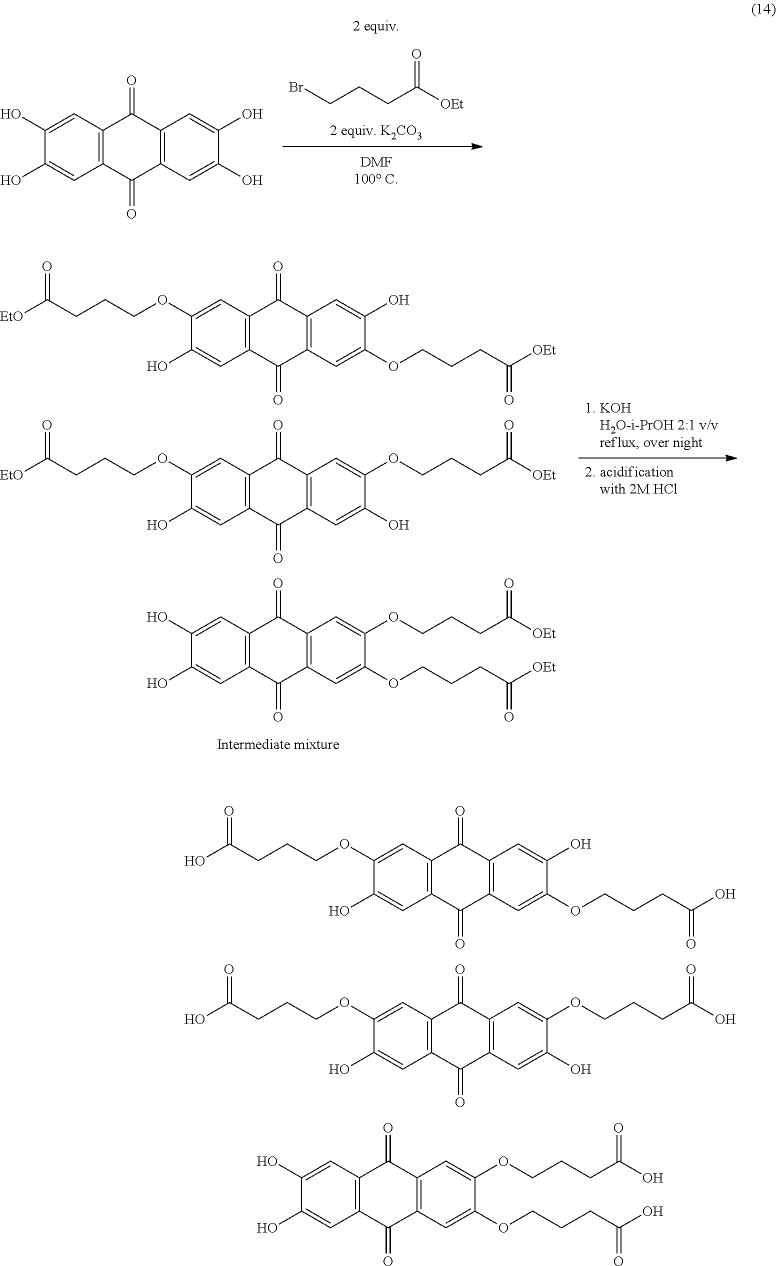

In a 1 L eggplant flask, 19.8 g (72.2 mmol) of 2,3,6,7-THAQ and 280 mL of DMF were placed. To this were added 19.9 g (144 mmol) of potassium carbonate and 20.7 mL (144mmol) of ethyl 4-bromobutanoate. The temperature rise was then started, and the mixture was stirred at 100° C. for 23 hours. After standing to cool, 150 mL of distilled water was added, and the precipitate was suction filtered. 6M hydrochloric acid was added to the filtrate with stirring until the pH was about 3 to 4, and the precipitate was collected by centrifugation and suction filtration. This precipitate was subjected to soxhlet extraction with chloroform. The extracted solution was vacuum concentrated to obtain 5.04 g of an intermediate mixture (14% yield).

5.15 g of the intermediate mixture was put in a 500mL eggplant flask, and 90 mL of isopropyl alcohol and 180 mL of distilled water were added. After adding 4.62 g (82.3 mmol) of potassium hydroxide, the temperature rise was started, and the mixture was heated and stirred at 60° C. for 20 hours. After standing to cool, 300 mL of distilled water was poured into a 1L beaker, and 2 M hydrochloric acid was added with stirring until the pH was less than 3. After stirring for 1 hour, the sediment was separated by centrifugation. The sediment was suction filtered while washing with distilled water and collected, and the residue was vacuum dried at 70° C. for 2.5 hours to obtain 3.71 g of the target substance (91% yield of mixture from the intermediate mixture).

### <Example 4>

2,6-MHMBEAQ was synthesized by the reactions represented by the following chemical reaction formulas (15) to (17). First, (2′-ethyl) hexyl 4-bromobutanate was synthesized according to the following procedure by reaction represented by chemical reaction formula (15).

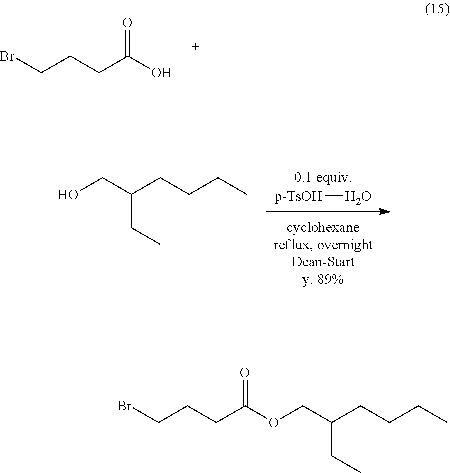

In a 200-mL eggplant flask, 8.56 g (51.3 mmol) of 4-bromobutanoic acid and 70mL of cyclohexane were placed. To this were added 7.40 mL (47.2 mmol) of 2-ethyl-1-hexanol and 0.98 g (5.14 mmol) of p-toluenesulfonic acid monohydrate, a Dean-Stark apparatus was attached, and the mixture was heated and refluxed for 21 hours. After standing to cool, the reaction solution was transferred to a separating funnel, 30 mL of cyclohexane and 50 mL of saturated sodium bicarbonate aqueous solution were added, and the two phases were separated. The organic phase was washed twice with 50 mL of saturated sodium bicarbonate aqueous solution and dried over anhydrous sodium sulfate. After removal of the drying agent by filtration, the product was vacuum concentrated and vacuum dried to obtain 11.8 g of a pale yellow oily product (89% yield in chemical reaction (15)).

Next, the pale yellow oily product, (2′-ethyl) hexyl 4-bromobutanate, was reacted with 2.6-DHAQ (Tokyo Chemical Industry Co., Ltd.) according to the following procedure by reaction represented by chemical reaction formula (16). In a 50mL eggplant flask, 1.20 g (4.99mmol) of 2,6-DHAQ and 19 mL of N-methyl pyrrolidone were placed. To this was added 0.52 g (3.78 mmol) of potassium carbonate, the temperature was raised to 95° C., 0.70 g of the pale yellow oily product was added, and the mixture was heated and stirred for 21 hours while maintaining the temperature at 95° C. After cooling with ice, 25 mL of distilled water was added to the reaction solution, and the separated viscous solid (0.61 g) was suction filtered, washed with water, and collected. The solid was dispersed in 25 mL of hexane, the sediment was collected by centrifugation, and the same procedure was repeated with another 25 mL of hexane to obtain 0.49 g of a pale yellow solid (22% yield in chemical reaction (16)).

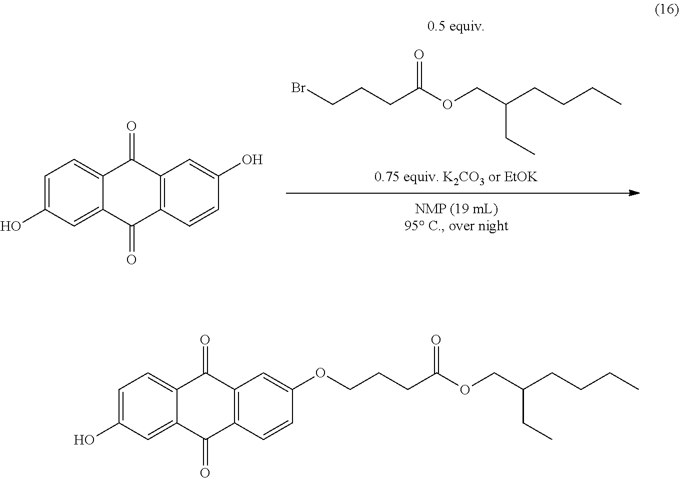 [SMILES: O=C1c2ccc(O)cc2C(=O)c2ccc(O)cc21 ; CCCCC(CC)COC(=O)CCCBr ; CCCCC(CC)COC(=O)CCCOc1ccc2c(c1)C(=O)c1ccc(O)cc1C2=O]

Next, 2,6-MHMBEAQ was synthesized according to the following procedure by reaction represented by chemical reaction formula (17). 0.294 g of the pale yellow solid was put in a 50mL eggplant flask with 10 mL of isopropyl alcohol and 20 mL of distilled water.

To this was added 0.211 g (2.68 mmol) of potassium hydroxide, the temperature rise was started, and the mixture was stirred at 60° C. for 20 hours. After standing to cool, 20 mL of distilled water was added, and 2 M hydrochloric acid was added with stirring until the pH was less than 3. At this time, a yellow sediment was dispersed throughout the solution. After stirring for 2 hours, the sediment was separated by centrifugation. The supernatant and sediment were separately suction filtered, and the residue was washed with distilled water. The solid was vacuum dried at 80° C. for 2 hours to obtain 0.206 g of 2,6-MHMBEAQ (94% yield in chemical reaction formula (17)).

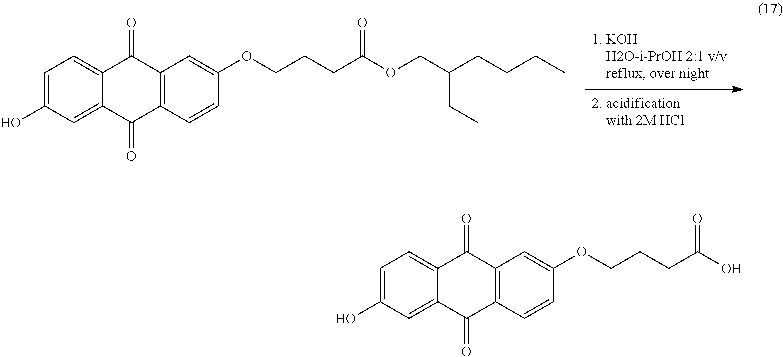 [SMILES: CCCCC(CC)COC(=O)CCCOc1ccc2c(c1)C(=O)c1ccc(O)cc1C2=O ; O=C(O)CCCOc1ccc2c(c1)C(=O)c1ccc(O)cc1C2=O]

The contents described in the above embodiments would be understood as follows, for instance.

[1] A method of producing an anthraquinone-based substance according to one aspect is a method of producing an anthraquinone-based substance represented by the following chemical formula:

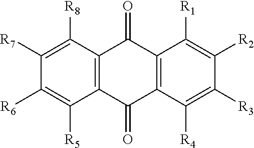

where at least one of the R₁ to R₈ is a hydroxy group, and at least one of the R₁ to R₈ is an alkoxy group, includes the steps of: preparing a starting material represented by the following chemical formula:

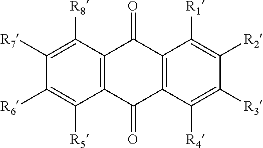

where at least two of the R₁′ to R₈′ are hydroxy groups; and reacting the starting material with an organic alkylating agent. The amount of the organic alkylating agent to be reacted with the starting material is more than or equal to 0.05 mol and less than n mol per 1 mol of the starting material, where n is the number of hydroxy groups contained in the starting material.

With the method of producing an anthraquinone-based substance of the present disclosure, by the reaction of more than or equal to 0.05 mol and less than n mol of the organic alkylating agent per 1 mol of the starting material, where n is the number of hydroxy groups contained in the starting material, hydroxy groups in the molecule of the starting material partially react with the organic alkylating agent, so that an anthraquinone-based substance with different substituents can be efficiently obtained.

[2] A method of producing an anthraquinone-based substance according to another aspect is the method of [1], in which R₂′ and R₆′ of the R₁' to R₈′ are hydroxy groups and remainder of the R₁′ to R₈′ is a hydrogen atom.

With this configuration, since 2,6-dihydroxyanthraquinone, which is industrially mass-produced, is used as the starting material, the production cost of the anthraquinone-based substance can be reduced.

[3] A method of producing an anthraquinone-based substance according to still another aspect is the method of [1], in which R₂′, R₃′, R₆′ and R₇′ of the R₁′ to R₈′ are hydroxy groups and remainder of the R₁′ to R₈′ is a hydrogen atom.

With this configuration, since 2,3,6,7-tetrahydroxyanthraquinone, which can be synthesized at high yield and thus is readily available, is used as the starting material, the production cost of the anthraquinone-based substance can be reduced.

[4] A method of producing an anthraquinone-based substance according to still another aspect is the method of any of [1] to [3], in which the organic alkylating agent is an alkyl carboxylic acid ester halide. The method includes the step of, after reacting the starting material with the alkyl carboxylic acid ester halide, hydrolyzing a reaction mixture with an alkali and treating the reaction mixture with an acid.

With this configuration, it is possible to efficiently obtain an anthraquinone-based substance with an alkoxy group containing a carboxy group attached.

[5] A method of producing an anthraquinone-based substance according to still another aspect is the method of any of [1] to [3], in which the organic alkylating agent is an alkyl carboxylic acid ester halide. The method includes the step of, after reacting the starting material with the alkyl carboxylic acid ester halide, hydrolyzing a reaction mixture in an alkaline aqueous solution.

With this configuration, an alkaline aqueous solution containing the anthraquinone-based substance is obtained, which can be used as-is in redox flow batteries as alkaline electrolyte.

[6] A method of producing an anthraquinone-based substance according to still another aspect is the method of any of [1] to [3], in which the organic alkylating agent is an alkyl carboxylic acid ester halide. The method includes the step of, after reacting the starting material with the alkyl carboxylic acid ester halide, hydrolyzing a reaction mixture with an alkali and vacuum concentrating the reaction mixture.

With this configuration, an alkaline aqueous solution containing the anthraquinone-based substance is obtained by vacuum concentration after hydrolysis in alkali, which can be used as-is in redox flow batteries as alkaline electrolyte.

[7] A method of producing an anthraquinone-based substance according to still another aspect is the method of any of [4] to [6], in which the alkyl carboxylic acid ester halide is an alkyl 4-bromobutanoate.

If the carbon chain of the alkyl carboxylic acid ester halide is short, E2 reaction may occur, causing the alkyl carboxylic acid ester halide to partially decompose and no longer contribute to the reaction. In contrast, with this configuration, since the carbon chain length of the butanoic acid backbone of alkyl 4-bromobutanoate is of an appropriate length, alkyl 4-bromobutanoate is difficult to decompose. Therefore, most of the alkyl 4-bromobutanoate added can contribute to the reaction, and the yield of the target product can be increased.

[8] A method of producing an anthraquinone-based substance according to still another aspect is the method of [7], in which water is added to a reaction solution after the hydrolysis and filtered, an acetic acid aqueous solution is added to a resulting filtrate and filtered, chloroform is added to a resulting residue and filtered, and a resulting filtrate is concentrated and dried.

With this configuration, it is possible to obtain an anthraquinone-based substance

with different substituents at high yield.

[9] A method of producing an anthraquinone-based substance according to still another aspect is the method of [7], in which hydrochloric acid is added to a reaction solution after the hydrolysis and filtered, tetrahydrofuran is added to a resulting residue and filtered, ethyl acetate is added to a solid obtained by concentrating and drying a resulting filtrate and filtered, chloroform is added to a solid obtained by concentrating and drying a resulting filtrate and filtered, methanol is added to a solid obtained by concentrating and drying a resulting filtrate and filtered, and a resulting filtrate is concentrated and dried.

With this configuration, it is possible to obtain an anthraquinone-based substance with different substituents at high yield.

[10] A method of producing an anthraquinone-based substance according to still another aspect is the method of any of [4] to [6], in which an alkyl group of an ester portion of the alkyl carboxylic acid ester halide has a carbon chain with four or more carbon atoms bonded together in a branched manner.

This configuration facilitates separation of anthraquinone-based substances in the

washing and extraction processes due to a large difference in solubility between an anthraquinone-based substance with different substituents and an anthraquinone-based substance with identical substituents, which are by-products.

[11] A method of producing an anthraquinone-based substance according to still another aspect is the method of any of [1] to [10], in which the starting material is 2,6-dihydroxyanthraquinone. The method further includes the step of synthesizing the 2,6-dihydroxyanthraquinone using 2,6-diaminoanthraquinone as a raw material.

With this configuration, by producing 2,6-dihydroxyanthraquinone as the starting material from 2,6-diaminoanthraquinone, which is less expensive than 2,6-dihydroxyanthraquinone, the production cost of the anthraquinone-based substance can be reduced.

1. A method of producing an anthraquinone-based substance represented by the following chemical formula: 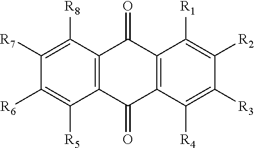
where at least one of the R₁ to R₈ is a hydroxy group, and at least one of the R₁ to R₈ is an alkoxy group,
the method comprising the steps of:
preparing a starting material represented by the following chemical formula: 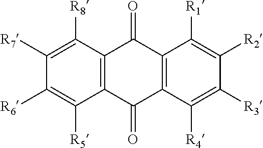
where R₂′, R₃′, R₆′ and R₇′at least two of the R₁′ to R₈′ are hydroxy groups and remainder of the R₁′ to R₈′ is a hydrogen atom; and
reacting the starting material with an organic alkylating agent,
wherein amount of the organic alkylating agent to be reacted with the starting material is more than or equal to 0.05 mol and less than n mol per 1 mol of the starting material, where n is the number of hydroxy groups contained in the starting material.

2. (canceled)

3. (canceled)

4. The method of producing the anthraquinone-based substance according to claim 1,
wherein the organic alkylating agent is an alkyl carboxylic acid ester halide, and
wherein the method comprises the step of, after reacting the starting material with the alkyl carboxylic acid ester halide, hydrolyzing a reaction mixture with an alkali and treating the reaction mixture with an acid.

5. The method of producing the anthraquinone-based substance according to claim 1,
wherein the organic alkylating agent is an alkyl carboxylic acid ester halide, and
wherein the method comprises the step of, after reacting the starting material with the alkyl carboxylic acid ester halide, hydrolyzing a reaction mixture in an alkaline aqueous solution.

6. The method of producing the anthraquinone-based substance according to claim 1,
wherein the organic alkylating agent is an alkyl carboxylic acid ester halide, and
wherein the method comprises the step of, after reacting the starting material with the alkyl carboxylic acid ester halide, hydrolyzing a reaction mixture with an alkali and vacuum concentrating the reaction mixture.

7. The method of producing the anthraquinone-based substance according to claim 4, wherein the alkyl carboxylic acid ester halide is an alkyl 4-bromobutanoate.

8. The method of producing the anthraquinone-based substance according to claim 7, wherein water is added to a reaction solution after the hydrolysis and filtered, an acetic acid aqueous solution is added to a resulting filtrate and filtered, chloroform is added to a resulting residue and filtered, and a resulting filtrate is concentrated and dried.

9. The method of producing the anthraquinone-based substance according to claim 7, wherein hydrochloric acid is added to a reaction solution after the hydrolysis and filtered, tetrahydrofuran is added to a resulting residue and filtered, ethyl acetate is added to a solid obtained by concentrating and drying a resulting filtrate and filtered, chloroform is added to a solid obtained by concentrating and drying a resulting filtrate and filtered, methanol is added to a solid obtained by concentrating and drying a resulting filtrate and filtered, and a resulting filtrate is concentrated and dried.

10. The method of producing the anthraquinone-based substance according to claim 4, wherein an alkyl group of an ester portion of the alkyl carboxylic acid ester halide has a carbon chain with four or more carbon atoms bonded together in a branched manner.

11. The method of producing the anthraquinone-based substance according to claim 1,
wherein the starting material is 2,6-dihydroxyanthraquinone, and
wherein the method further comprises the step of synthesizing the 2,6-dihydroxyanthraquinone using 2,6-diaminoanthraquinone as a raw material.
